(19) United States
(12) Patent Application Publication
(10) Pub. No.: US 2018/0240537 A1
(43) Pub. Date: Aug. 23, 2018

(54) GUIDED STRUCTURED REPORTING

(71) Applicant: KONINKLIJKE PHILIPS N.V., EINDHOVEN (NL)

(72) Inventors: MERLIJN SEVENSTER, CHICAGO, IL (US); THOMAS ANDRE FORSBERG, HAYWARD, CA (US)

(21) Appl. No.: 15/553,300

(22) PCT Filed: Mar. 9, 2016
(86) PCT No.: PCT/IB2016/051316

§ 371 Date: Aug. 24, 2017

Related U.S. Application Data
(60) Provisional application No. 62/135,230, filed on Mar. 19, 2015.

(51) Int. Cl. G16H 15/00; G16H 50/20

(52) U.S. Cl. CPC G16H 15/00; G06F 3/0482; G16H 50/20

(57) ABSTRACT

A guided structured reporting apparatus (10) that enables clinicians to select report elements and generate a structured report (56), offering novel and improved structured reporting solutions that improves report (56) accuracy and precision, and expedites the generation of such a report (56). One or more processors (18) receive physiological information, generate a display, generate and display suggested finding codes (48) for adoption in the structured report (56), and generates and displays a structured report (56) based on the adopted finding codes (40).

### FIELD

The following relates to patient diagnostic and reporting systems that provide improved structured reports. It finds particular application in conjunction with clinical informatics, especially cardiovascular informatics. However, it is to be appreciated that it will also find application with respect to other usage scenarios, and is not necessarily limited to the aforementioned application.

### BACKGROUND

Currently, tools exist, such as the multi-modality tool Philips Xcelera™ which allows clinicians to select report elements and generate a report. A feature of that product is that it allows for reporting in terms of pre-defined finding codes (FCs). Each finding code includes a code component and a textual component. However, this tool requires one-by-one selection of finding codes (FCs), which in turn requires every clinician to navigate multiple menus and submenus prior to obtaining a final report. Generally, once physiological information, including patient imaging data such as echocardiograms, has been generated, clinicians review these images, conduct dictation, and generate a report. However, because the current reporting tools require one-by-one selection of finding codes, this process is time-consuming and may result in reporting errors, such as leaving aspects of a report unaddressed or overlooked. This is because any given structured reporting system might support an average of 1000 different finding codes, while a final report will only include approximately 30 finding codes.

Thus, a serious problem exists in current technology that results in reporting that is incomplete, tedious, and time-consuming.

### SUMMARY

In accordance with one aspect, a guided structured reporting apparatus is provided for guiding a clinician to address unaddressed aspects of the report. The apparatus comprises a workstation including a display device configured to display physiological information, and an input configured to receive one or more inputs from a clinician; one or more computer processors connected to the workstation, the one or more computer processors being configured to receive physiological information, generate a display preliminary report on the display device, receive and display finding codes from the input selected by the clinician for inclusion in the preliminary report, generate and display suggested finding codes for potential inclusion in the preliminary report, and update the displayed finding codes on the display device based on at least one of the clinician selected finding codes and physiological information.

In accordance with another aspect, a method is provided for guiding a clinician to address unaddressed aspects of a patient report. The method comprises receiving physiological information at a workstation, generating a display of a preliminary report on a display device using one or more processors, generating and displaying one or more suggested finding codes for the clinician to select among using one or more processors, receiving a clinician's selection of one or more of the suggested finding codes from a user input and incorporating the selected finding code in the preliminary report, and updating the displayed finding codes based on the one or more of the clinician selected finding codes and physiological information.

One advantage of these aspects is that they are compatible with the existing structured reporting systems.

Another advantage resides in improving effectiveness and accuracy of patient reporting.

Another advantage resides in providing a reporting workstation with an improved user interface.

Another advantage resides in providing a reporting workstation facilitating more accurate and rapid reporting.

Another advantage resides in improving the completeness of patient reporting, and ensuring that there are no gaps in the patient report.

Another advantage resides in improving clinical care outcomes and quality of care.

Another advantage resides in reducing reporting errors and oversights.

Another advantage resides in facilitating the presentation of finding codes and minimizing the need for locating and navigating myriad menus and submenus.

Another advantage resides in providing a guided structured reporting apparatus capable of recommending physiological parameter measurements, such as in order to facilitate patient risk assessment and to ensure unaddressed aspects of a report are addressed.

Still further advantages of the present invention will be appreciated to those of ordinary skill in the art upon reading and understanding the following detailed description. It is to be appreciated that none, one, two, or more of these advantages may be achieved by a particular embodiment.

### BRIEF DESCRIPTION OF THE DRAWINGS

The invention may take form in various components and arrangements of components, and in various steps and arrangement of steps. The drawings are only for purposes of illustrating the preferred embodiments and are not to be construed as limiting the invention.

FIG. 1 diagrammatically illustrates a guided structured reporting apparatus according to one embodiment.

FIG. 2 illustrates a display of a guided structured reporting apparatus according to one embodiment.

FIG. 3 diagrammatically illustrates a display of a guided structured reporting apparatus according to another embodiment.

FIG. 4 diagrammatically illustrates a display on a display device of a guided structure reporting apparatus.

FIG. 5 illustrates a structured reporting pane according to one embodiment.

FIG. 6 illustrates a suggested finding code pane according to one embodiment.

FIG. 7 illustrates a suggested finding code pane according to another embodiment.

FIG. 8 illustrates a narrative report according to one embodiment.

FIG. 9 illustrates a display of a guided structured reporting apparatus with a narrative report as an element of the display.

FIG. 10 diagrammatically illustrates a display on a display device of a guided structured reporting apparatus with a narrative report as an element of the display.

FIG. 11 illustrates a method flow chart or means diagram for the guided structured reporting of physiological information by using a guided structured reporting apparatus.

### DETAILED DESCRIPTION

The present disclosure is directed to apparatuses and methods for the guided structured reporting of patient physiological information which in various embodiments provide various advantages such as reducing reporting errors, guiding the clinician to address potentially overlooked aspects of a patient report, and improving efficiency of the reporting workstation user interface.

The present disclosure is further directed to apparatuses and methods generating and suggesting physiological parameters to be considered and adopted into a patient report.

The present disclosure is further directed to apparatuses and methods incorporating and displaying suggested physiological parameters to be considered and adopted into a patient report that reduces the burden placed on clinicians and other users of the apparatuses and methods when navigating interface the disclosed apparatuses and methods.

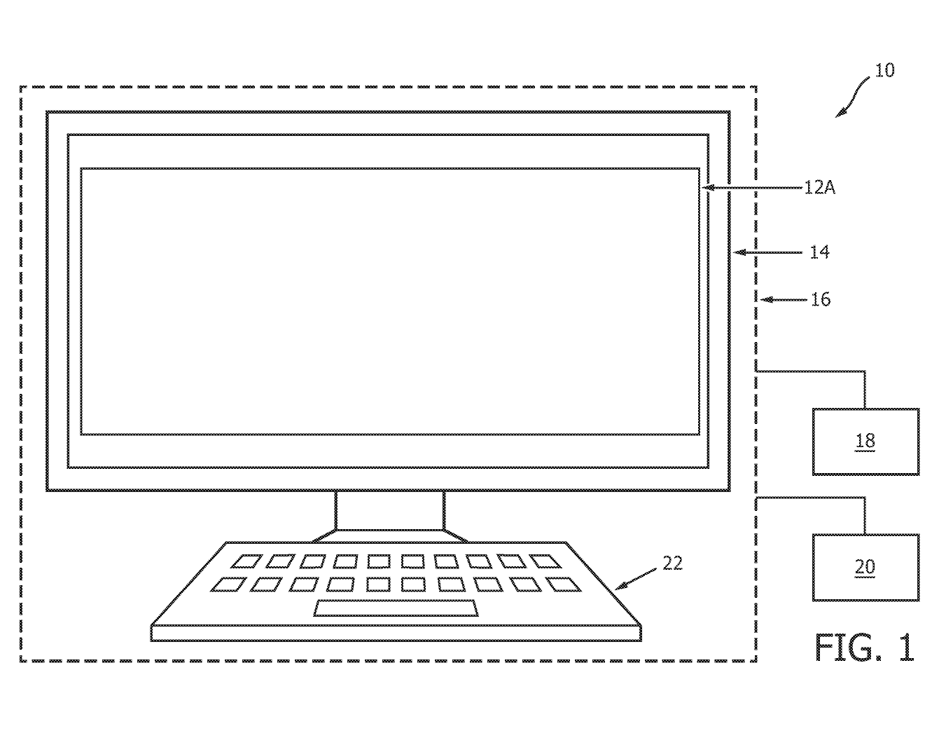

FIG. 1 diagrammatically illustrates a guided structured reporting apparatus 10 according to one embodiment. According to this embodiment, the apparatus 10 includes a workstation 16 and one or more processors 18 connected to the workstation 16. The workstation includes a display device 14 configured to display physiological information, and at least one user input device 22 (e.g., the illustrative keyboard, and/or a mouse or other pointing device, and/or a touch soverlay on the display 14, or so forth) configured to receive one or more inputs. The one or more processors 18 connected to the workstation 16 are configured to at least: receive physiological information; receive one or more inputs from the input 22; generate a display 12A presented on the display device 14; generate one or more suggested finding codes 48; and update the display 12A on the display device 14 based on inputs from the input device 22 or in response to receiving additional physiological information. It will be appreciated that the one or more processors 18 may be separate from the user interfacing components 14, 22 (e.g. a web-based server or cloud computing system) and/or may be integrally built (e.g. a notebook or desktop computer in which a processor is integral to the computer) Additionally, in some embodiments, the workstation 16 of the apparatus 10 can be connected to at least one memory system 20 in which patient information, including physiological information is stored. The memory 20 can also store one or more suggested finding codes and newly created patient reports. In some embodiments, the memory 20 includes a clinical information system.

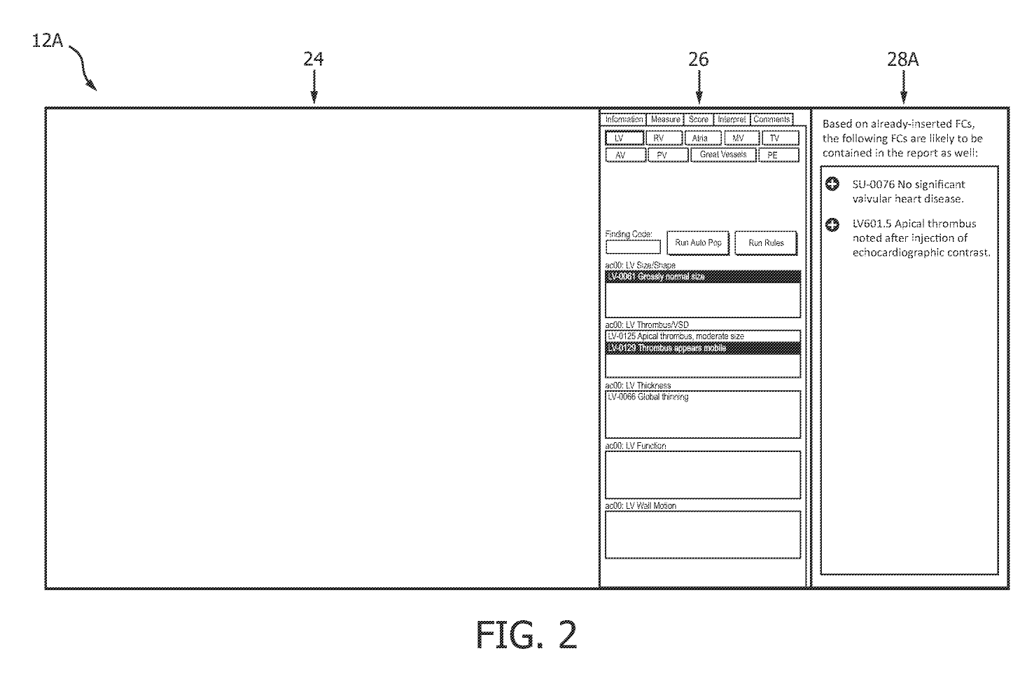

FIG. 2 illustrates an enlarged view of the display 12A of FIG. 1. As seen in FIG. 2, the one or more processors 18 of the apparatus 16 are configured to generate the display 12A including at least a structured reporting pane 26 and a finding code suggestion pane 28A. The display 12A also includes an imaging pane 24 (or, more generally, a patient data presentation or rendering or summarization pane) in which additional patient physiological information, such as echocardiogram images, can be reviewed. In particular embodiments, the finding code suggestion pane 28A may be a persistent window. In other words, in particular embodiments, the finding code suggestion pane 28A may always be an element of the display 12A in the apparatus 16.

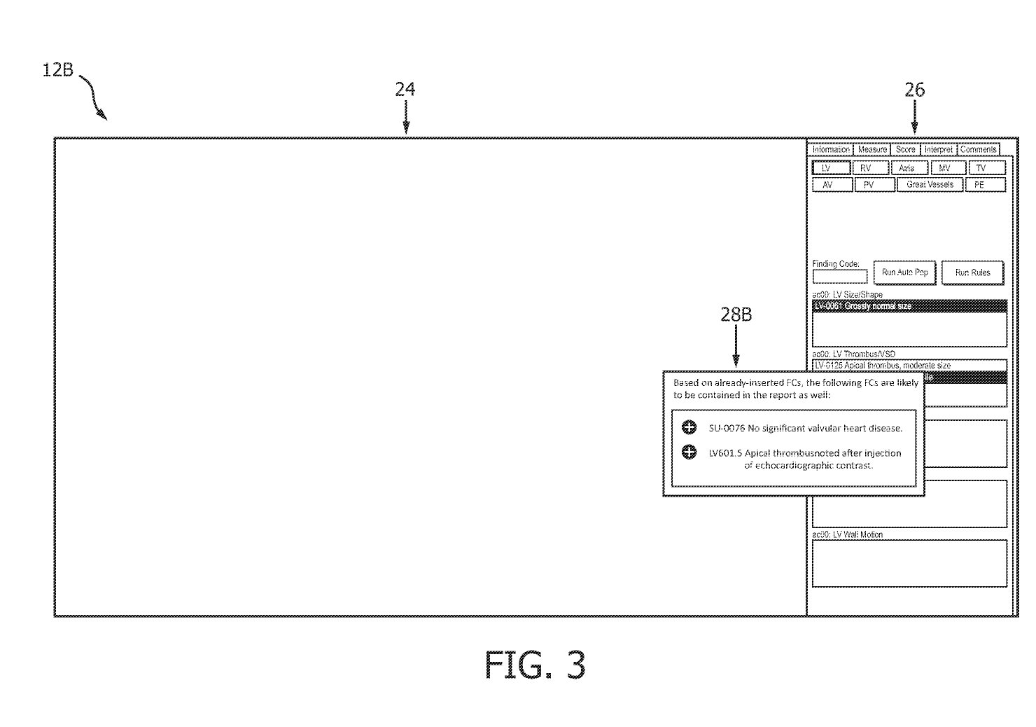

In alternative embodiments, as seen in FIG. 3, the one or more processors 18 of the apparatus 16 are configured to generate a display 12B on the display device 14 including at least a structured reporting pane 26 and a finding code suggestion pane 28B, wherein the finding code suggestion pane 28B is not a persistent window. In other words, in particular embodiments, the finding code suggestion pane 28B may be a transient window, and only be generated in the display 12B after receiving some input from the user via the one or more user input devices 22.

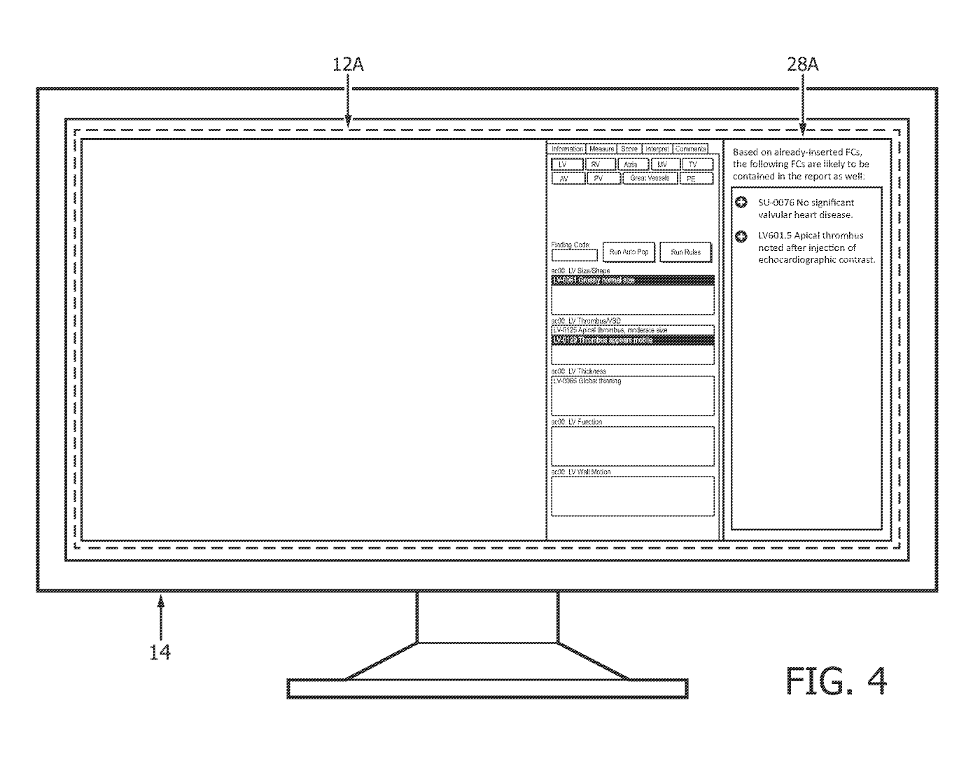

As seen in FIG. 4, the one or more processors 18 of the apparatus 16 are configured to generate the display 12A on the display device 14 of the apparatus 10. In particular embodiments, the display 12A may be a display 12A as seen in FIGS. 2 and 3, while in other embodiments, the display 12A may not include an imaging pane 24.

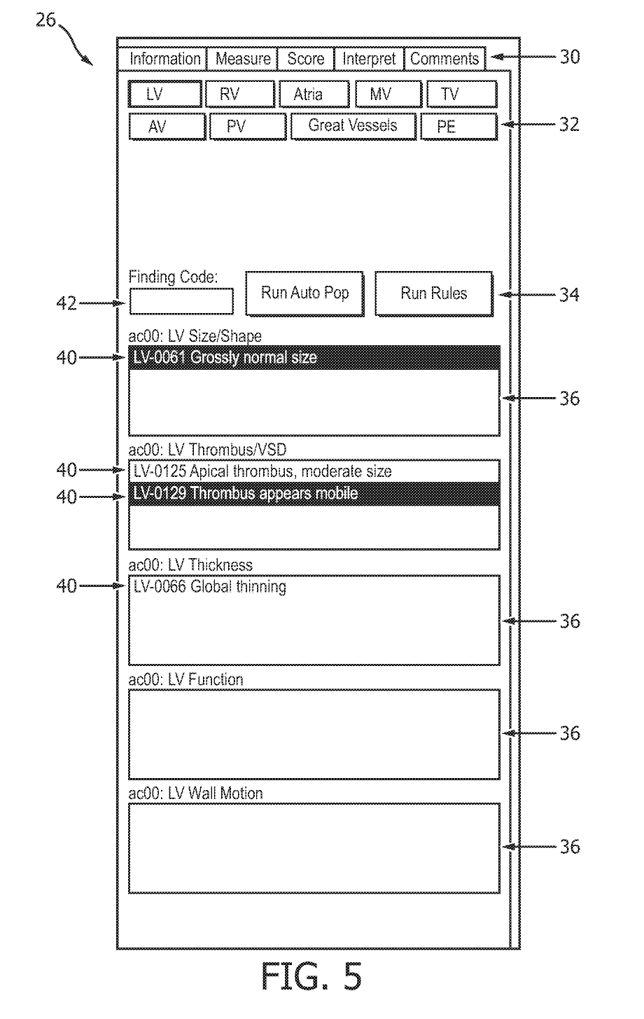

FIG. 5 illustrates a structured reporting pane 26 according to one embodiment. In particular embodiments, the reporting pane 26 may include an anatomical submenu 32 and aspect submenus 36. An anatomical submenu 32 and an aspect submenu 36 may include a variety of options depending on the particular application of the invention. In particular embodiments, such as cardiovascular applications, an anatomical submenu 32 may include options for “LV” (left ventricle), “RV” (right ventricle), “Atria”, “MV”, “TV”, “AV”, “PV”, “Great Vessels”, and “PE”. In particular embodiments, such as cardiovascular applications, an aspect submenu 36 may include aspects such as “Size/Shape”, “Thrombus/VSD”, “Thickness”, “Function”, and “Wall Motion”. In particular embodiments, the aspect submenu 36 options may vary across different anatomical submenu 32 options, and thus may change depending on which anatomical submenu 32 option is currently displayed.

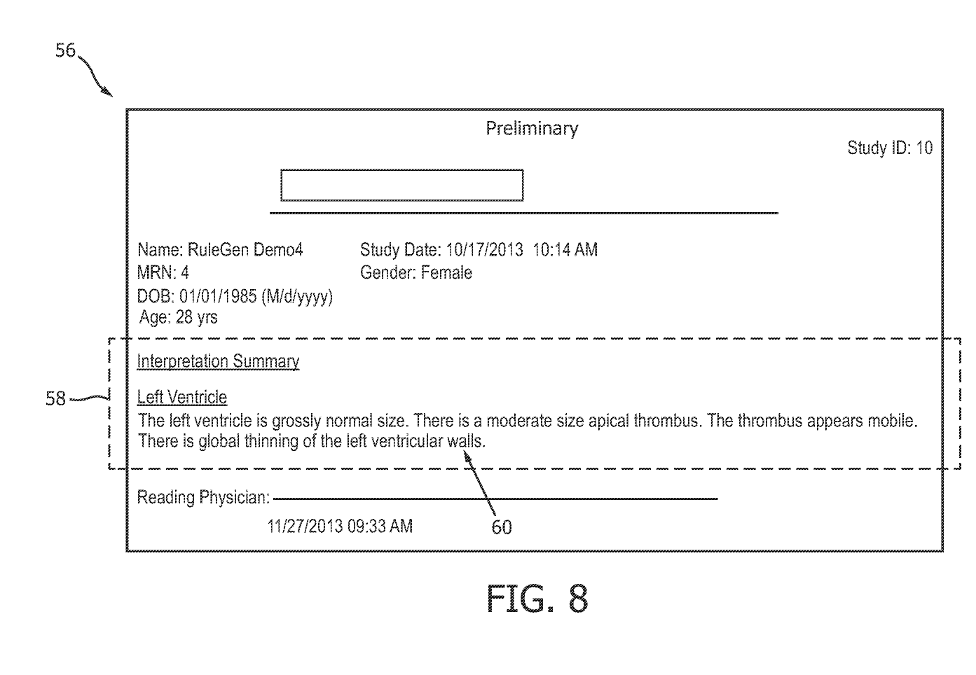

In particular embodiments, as seen in FIG. 5, the reporting pane 26 may also include one or more finding code generation buttons 34, one or more drop-down menus, and an option to search for finding codes 42. In particular embodiments, as also seen in FIG. 5, the reporting pane 26 preferably also includes one or more adopted finding codes 40. Adopted finding codes 40 are used to generate a narrative report 56 (e.g. FIG. 8).

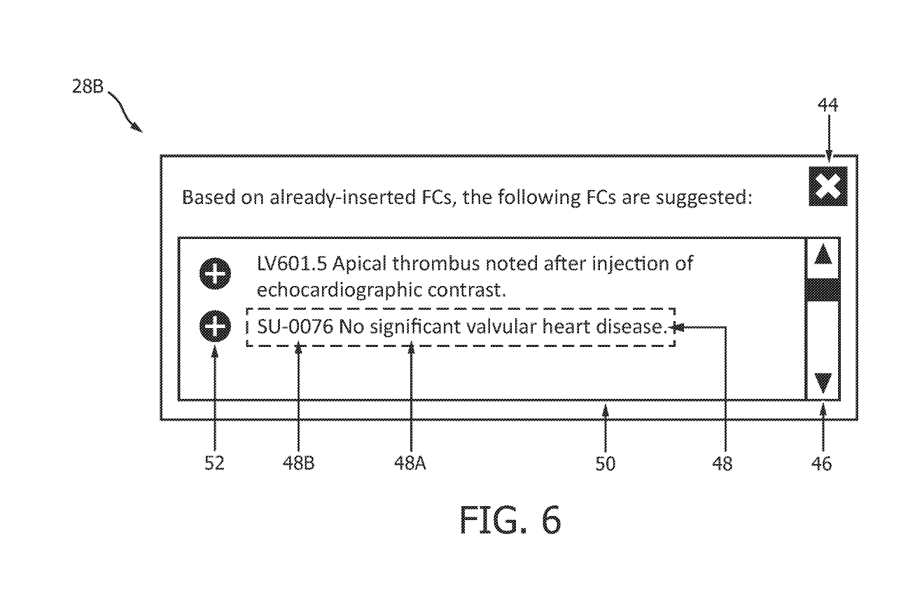

FIG. 6 illustrates a drop-down menu suggested finding code pane 28B according to one embodiment. In particular embodiments, a suggested finding code pane or a suggestion pane 28B may include one or more suggested finding codes 48 and one or more adoption buttons 52 corresponding to the one or more suggested finding codes 48. In particular embodiments, a suggestion pane 28B may be a transient suggestion pane 28B, which is only generated by the one or more processors 18 on the display 12B after receiving an input from an input 22. In particular embodiments, the suggestion pane 28B may have an exit or close button 54, wherein upon receiving an input from the input 22, the one or more processors 18 removes the suggestion 28B from the display. In other words, in particular embodiments, a display 12B, contains a suggestion pane 28B even if the suggestion pane 28B is not always visible or displayed in the display 12B.

In particular embodiments, the suggested finding codes 48 and adoption buttons 44 may be contained in a sub-window 50 of the suggestion pane 28B. Additionally, in particular embodiments, the suggestion pane 28B may contain a scroll-bar 46, wherein the one or more processors 18 are configured to scroll through lists of suggested finding codes 48 within the suggestion pane 28B upon receiving input from the input 22.

In particular embodiments, each finding code 40 and suggested finding code 48 may include a textual component 48A and a code component 48B. Finding codes 40, 48 may be at least one of several different types of finding codes. In particular embodiments, a finding code 40, 48 may be at least one of: a binary statement finding code, which are either true or false (e.g., “The LV is severely dilated.”); a multiple-choice statement finding code, in which a clinician or other user may select one of several predetermined options (e.g, “Mitral valve area by pressure half-time is . . . ” with options to select for “<100 msec”, “between 100 msec and 180 msec”, and “greater than 180 msec”); and a measurement statement finding code, in which a manually created measurement is used to complete a evaluation statement regarding a particular anatomical aspect.

As seen in FIG. 6, one or more processors 18 of the apparatus 10 may be configured to generate and display one or more suggested finding codes 48 for a variety of anatomies and aspects of anatomies. In other words, in some embodiments, the suggestion pane 28B may include suggested finding codes 48 that are related to and not related to the currently displayed anatomical submenu.

In the following, two illustrative embodiments are described that are suited for suggesting finding codes 48. The first is most suitable in clinical settings where large amounts of physiological information, such as from a database of structured reports for different patients, are not available. The second is most suitable in clinical settings where large amounts of physiological information, such as from a databased of structured reports for different patients, is available. However, these embodiments are not limited to such situations and may be performed in many different clinical environments. Moreover, the two illustrative embodiments may occur concurrently.

Furthermore, according to the present disclosure, either or both of the illustrative embodiments are able to (1) provide finding code suggestions 48 that are implied by the finding codes 40 contained in the current report 26, and/or (2) provide finding code suggestions 48 that are most informative given the finding codes 40 contained within the current report 26. Thus, either or both of the illustrative embodiments may allow for the adoption of overall impression statements (i.e., suggested finding codes 48) given concrete finding codes 40 already contained within the report 26, and may allow for the detection of anatomies 32 and aspects 36 that require further completion (e.g., if a clinician or other user of the apparatus 10 has fully specified all aspects 36 of the left ventricle anatomy 32, the illustrative embodiments will suggest finding codes 48 for adoption in anatomies 32 and aspects 36 that have not be addressed).

The foregoing displays are generated by a combination of a current report contents demon in the form of an engine that tracks all finding codes contained in the current study. This demon updates every time the contents of the report change. A finding code engine suggests the one or more finding codes for adoption. The user interface engine presents the one or more suggested finding codes to the user as shown in the displays discussed above. The current report demon can be implemented using standard application programming interface (API) methods. The finding code suggestion engine can be provided in various ways. In one embodiment, the finding code suggestions are selected based on the finding codes contained in the current (preliminary) report that the clinician is currently preparing. In another embodiment, the finding codes that are most important relative to the finding codes contained in the current (preliminary) report are suggested.

The first embodiment allows the insertion of overall impression statements given concrete findings. The second embodiment detects what aspects need further attention. For example, if the user has fully specified all values regarding the organ being analyzed. The system will automatically start suggesting the adoption of finding codes in areas that have not been addressed. For example, if all the values regarding the left ventricle have been fully specified, the system will start suggesting the adoption of finding codes in other areas that have not been addressed, such as the right ventricle.

When used in unison, these two embodiments can drive the entire structured reporting process so as to minimize the need for locating and navigating to sub-windows in which new finding codes need to be entered. The second type of functionality searches for the finding codes that carry the most informational value and suggest such codes in order to assist the clinician in covering all aspects of the exam's interpretation. The first type of functionality searches for finding codes that are implemented by the finding codes that have already been entered. The combination of these two functionalities facilitates avoiding gaps in the report.

In one embodiment, the finding codes are suggested using a rule-driven implementation in which a set of background rules are used for making suggestions. In another embodiment, the finding code suggestions are data driven. Suggestions are made based on statistics or other types of values derived from a database of prior reports.

The first illustrative embodiment pertains to a rule-driven implementation of suggesting finding codes 48 for display in a guided structured reporting apparatus 10. Generation of the suggested finding codes 48 can be performed by using a set of background rules used for making suggestions. According to this illustrative embodiment, the set of rules models the correlations between pre-existing finding codes. For example, a set of rules might be summarized according to the following:
If {at least one, all} of the finding codes [A₁, A₂, . . . , Aₙ] are in the current report R, then suggest {at least one, all} of the finding codes [S₁, S₂, . . . , Sₙ] for adoption in the current report R.

According to the first illustrative embodiment, the set of background rules used for generating suggested finding codes 48 as described above contains quantifier values for the finding codes available, including any pairwise combinations of finding codes, and evaluates determines whether the existing finding codes 40 imply other finding codes that should be suggested 48. These quantifier values may not be known, but may be estimated by retrospectively analyzing past patient data, and may be fine-tuned manually.

The second illustrative embodiment pertains to a data-driven implementation of suggesting finding code 48 for display and for adoption in a guided structured reporting apparatus 10. Generation of the suggested finding codes 48 can be performed by examining retrospective physiological information to determine the most similar prior sets of finding codes 40 and the likely next entered finding code. This process is expedited by determining the information value of potential finding codes such that high information value finding codes are suggested and used in the report. In some embodiments, one or more processors 18 are configured to generate suggested finding codes 48 by examining contents of the memory system 20 such as a database of retrospective echocardiogram reports (e.g. electrocardiogram reports that have been prepared for past patients), and suggest finding codes 48 that always occur (or frequently occur, e.g. above some specified threshold) given that another set of one or more finding codes 40 occur. In one approach, every finding code is regarded as a random variable that can take values from a discrete and finite domain: binary statement finding codes take values from the domain {true, false}; multiple-choice finding codes take values from their set of options; and measurement finding codes take values from the domain {25^{th} percentile, 50^{th} percentile, 75^{th} percentile}.

Thus, according to the second illustrative embodiment, if the current report contained finding codes [A₁, A₂, . . . , Aₙ] 40, then the one or more processors 18 will generate suggested finding codes [B] 48 for which the conditional probability:

P(B|A₁, . . . , Aₙ)≥θ

for a threshold, θ. To ensure the significance of the relation, particular embodiments may impose that the number of reports containing the set of finding codes [A₁, A₂, . . . , Aₙ] 40 and [B] exceeds a certain fixed or statistically determined threshold.

In particular embodiments of the second illustrative embodiment, the one or more processors 18 may generate suggested finding codes 48 based on the information value, or conditional entropy, of a finding code [B] given existing finding codes [A₁, A₂, . . . , Aₙ] 40. For example, in some embodiments of the second illustrative embodiment, the one or more processors 18 may generate suggested finding codes 48 by excluding finding codes that have a low information value based on the current finding codes 40 according to the following:

H(B|A₁, . . . , Aₙ)≤χ

for a threshold χ, where H is the conditional entropy of the finding code B given the set of adopted finding codes [A₁, A₂, . . . , Aₙ] 40. If the information value of B is below χ, then the finding code B carries little information per se and/or given the values of existing finding codes [A₁, A₂, . . . , Aₙ] 40. In other embodiments, the one or more processors 18 may generate suggested finding codes 48 by determining whether a finding code has a high information value given the finding codes 40 currently contained in the report according to the following:

H(B|A₁, . . . Aₙ)≤λ

for a threshold, λ.

According to either illustrative embodiment, the one or more processors 18 may update the one or more suggested finding codes 48 every time the contents of the report (i.e., the adopted finding codes 40) are altered. Additionally, the one or more processors 18 may be configured in either illustrative embodiment to sort and display the list of suggested finding codes 48 based upon the suggestion code's probability or conditional entropy. In particular embodiments, the one or more processors 18 may limit the number of suggested finding codes 48 displayed or generated at one time. In particular embodiments, the one or more processors 18 may be configured to display only those suggested finding codes 48 that are anatomically related to the anatomical region currently displayed. For example, in some embodiments, the suggestion pane 28A may only contain suggested finding codes 48 anatomically related to the left ventricle if “LV” is currently displayed in the report pane 26, as seen in FIG. 5.

The user interface engine preferably presents the suggested finding codes to the user in unambiguous, uncluttered ways. In one embodiment, the user interface engine presents the suggestions in a separate panel as a pop-up or background window 28B (see FIG. 3) in which the suggestions are refreshed every time the contents of the report are altered. In the other embodiment, the suggested finding codes are displayed in a native structured reporting window (e.g. window 28A of FIG. 2) for selection. These approaches provide the clinician with suggestions, but do not actually modify the report being prepared by the clinician in other words, the clinician is solely responsible for the content of the report, while the suggestions provided in the unambiguous window 28A or 28B are assistive in nature.

When there are multiple finding codes, the interface optionally sorts them based on the probability and/or entropy of the scores found by the binding code suggestion engine, in order to display the “most likely” finding codes at the top of the list or otherwise emphasized. If the number of suggested finding codes is too large to fit into the window 28A or 28B, the interface engine can clip off (i.e. not display) the least probable end of the list.

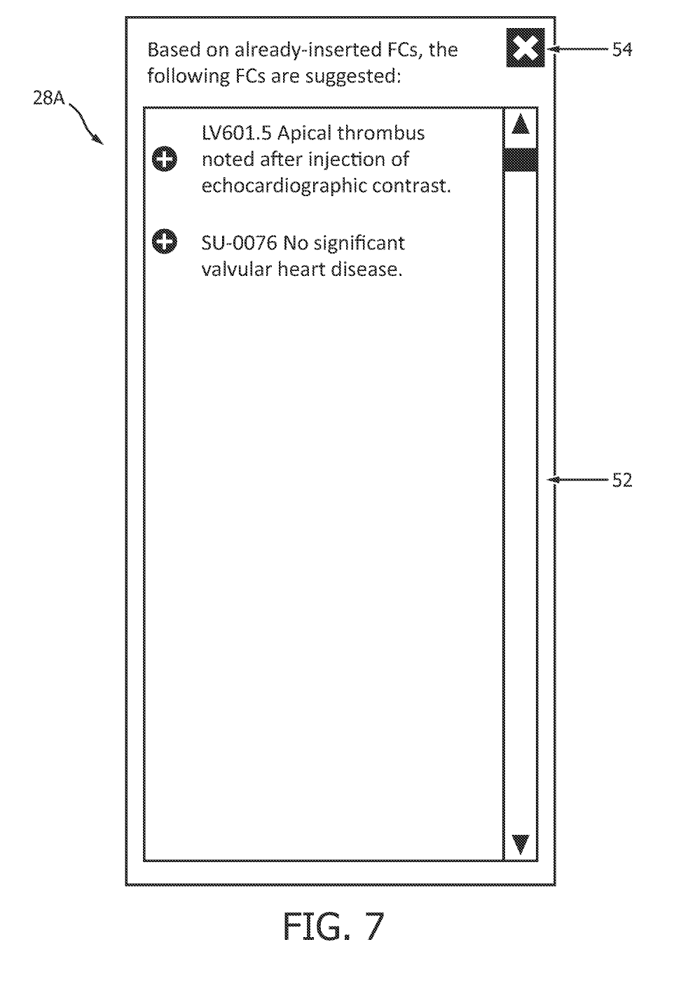

The user interface can also explain the mode of functionality that is supported by the suggestion (that is, the rationale by which the suggestion is being made). In the first functionality category, the interface could communicate this mode with a prefix such as “based on the already inserted finding codes, the following finding codes are likely to be contained in this report as well.” In the second functionality category, the interface could communicate with a prefix such as: “please specify the status of finding code A, which is optimally clinically informative according to our analysis.” In a more advanced embodiment, this engine can display only the suggested finding codes that are anatomically related, i.e., can be selected in the anatomical window, e.g., left ventricle as illustrated in FIG. 7.

FIG. 7 illustrates an enlarged view of the illustrative suggested finding code pane 28A. In particular embodiments, the suggestion pane 28A may be a persistent suggestion pane, as seen in FIG. 2. As seen in FIG. 7, one or more processors 18 are configured to generate the suggestion pane 28A that, unlike the transient suggestion pane 28B in FIG. 6, may always be displayed in the display 12A.

FIG. 8 illustrates a narrative report according to one embodiment. In particular embodiments, the one or more processors 18 operating in conjunction with the display device 14 and the one or more user interface devices 22 are configured to enable the clinician to generate a narrative report 56 based on the finding codes 40 that have been adopted into the current report. In particular embodiments, a narrative report contains an interpretation summary section 58, in which one or more anatomies 32 may be summarized in sub-sections 60. In particular embodiments, the one or more processors 18 display only the textual components of the adopted finding codes 48 within the interpretation summary section 58 and the anatomical sub-sections 60.

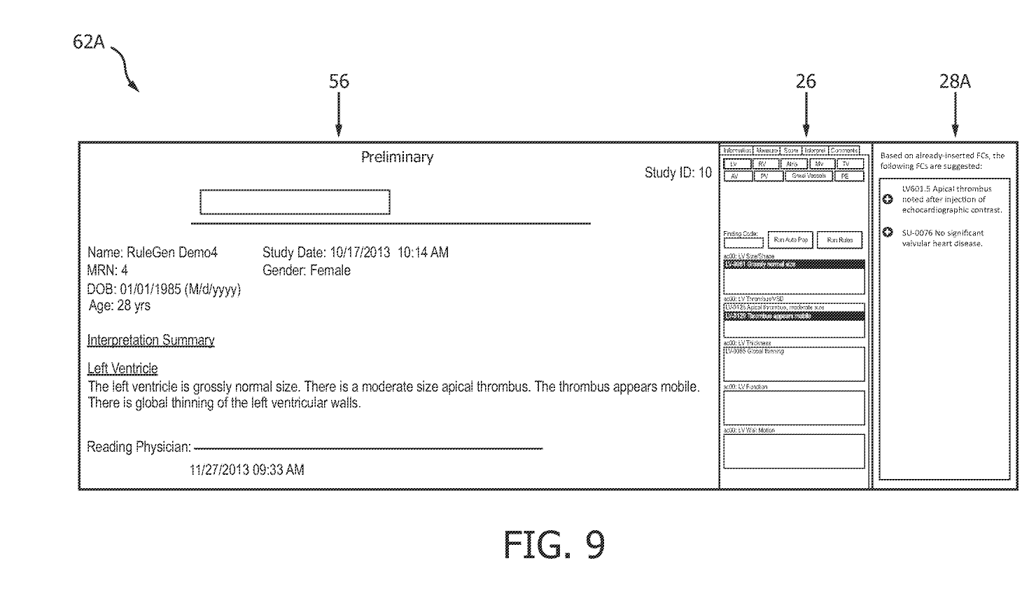

FIG. 9 illustrates a display of a guided structured reporting apparatus with a narrative report as an element of the display. As seen in FIG. 9, the one or more processors may be configured to generate a display 62A in which a narrative report 56 is displayed. In some embodiments, the narrative report 62A may be displayed rather than displaying an imaging panel 24. In other words, the narrative report 56 may be displayed even if an imaging panel 24 is not also displayed. However, like in FIGS. 2 and 3, the display 62A may include a reporting pane 26 and a persistent suggestion pane 28A (as shown in FIG. 9) or a transient suggestion pane 28B (e.g. see FIG. 3).

In another embodiment, instead of a separate window for the suggested field codes (either attached or free floating), the suggested field codes are automatically inserted in the report being prepared, such as by filling in boxes in the report. To denote that the field codes are suggested, they are “greyed out”, semitransparent, or otherwise visually denoted as suggested rather than adopted. If the clinician wants to adopt one of the greyed out suggested field codes, double-clicking the suggested field code adds it to the report in the same text format as the other adopted field codes. In one variation on this embodiment, there is an upper limit to the number of greyed out or suggested field codes which are presented to avoid overwhelming the clinician, e.g. 2 field codes per panel. More specifically, the 2 most relevant (by any chosen metric) suggested field codes appear greyed out. The clinician can easily double click on the one(s) to be added to the report. Once one suggested field code is adopted, a new greyed out suggested field code is inserted in the place in the report appropriate to the new field code to replace the adopted field code.

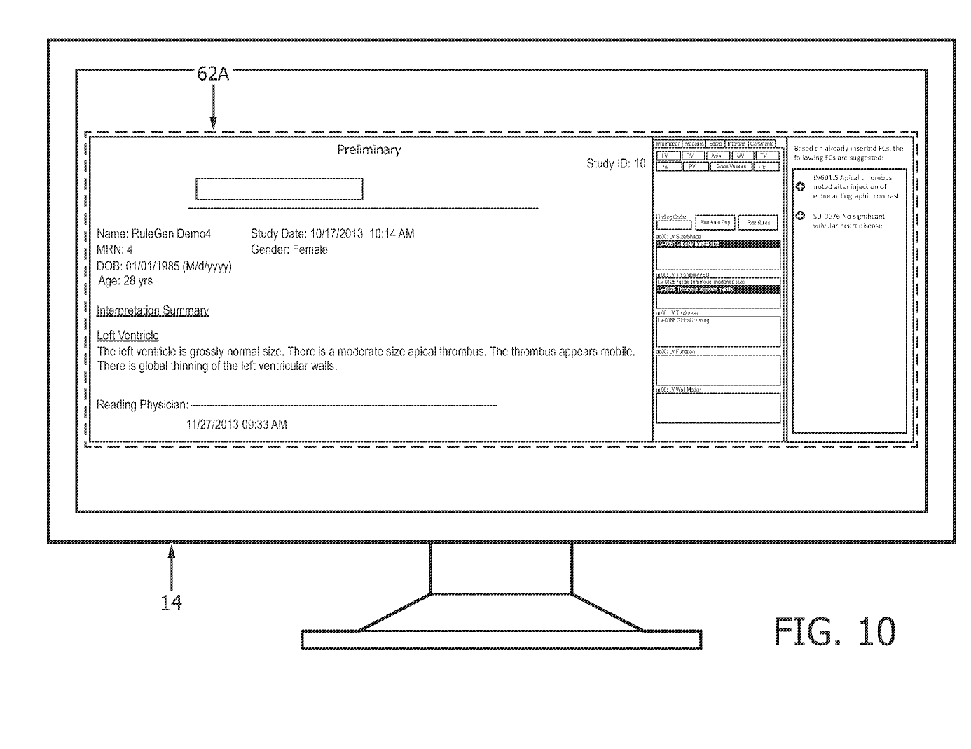

FIG. 10 diagrammatically illustrates a display on a display device of a guided structured reporting apparatus with a narrative report as an element of the display. In particular embodiments, the one or more processors may generate a display 62A including a narrative report 56 on a display device 14 of the apparatus 10.

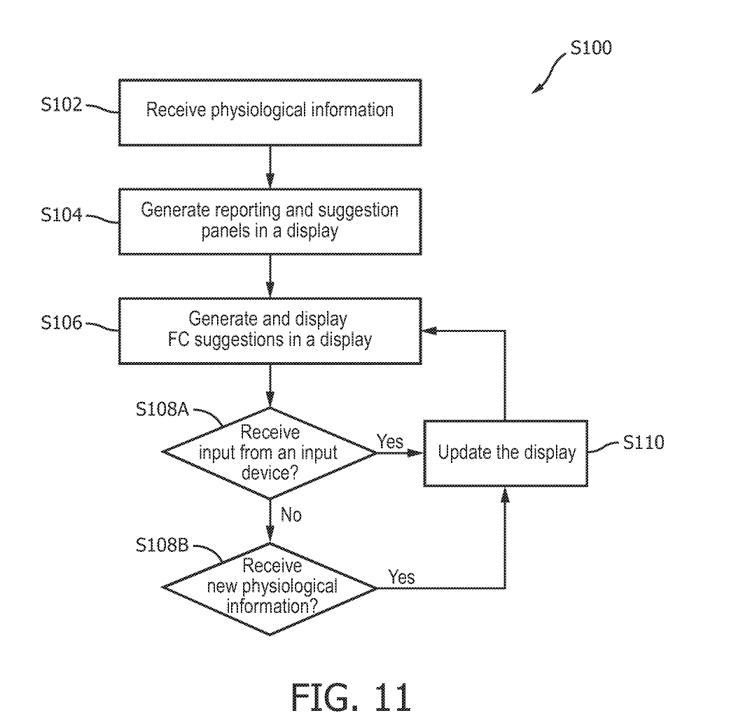

FIG. 11 illustrates a method S100 flow chart for the guided structured reporting of physiological information by using a guided structured reporting apparatus. In a first step S102, physiological information is received at a workstation 16. In particular embodiments, the physiological information may include at least one of patient physiological imaging information, patient records, patient finding codes 48, patient structured reports 58, and stored rules for suggesting finding codes. Using this physiological information, the one or more processors 18 generate a display 12, 62 on the display device 14 of the workstation 16 in a second step S104. In particular embodiments, the display may include at least one of a reporting panel 26 and a suggestion panel 28. In some embodiments, as described above, a transient suggestion panel 28B may always be visible. In a third step S106, finding code (FC) suggestions are generated by the one or more processors as described herein based on the physiological information. As the clinician prepares the report, he or she inputs data in step S108A, in which the one or more processors 18 receive user input via the user input device(s) 22, for example in the form of keyboard entry, selections using a mouse or other pointing device, or so forth. In a forth step S108B, the one or more processors 18, which are configured to receive physiological information, may receive new physiological information. In particular embodiments, new physiological information may include new patient imaging data. In step S110, the one or more processors 18 update the display 12/62 based upon the input received in the third step S108A or the information received in the forth step S108B. In particular embodiments, updating the display may include at least one of generating and displaying a narrative report based upon the finding codes 40 adopted by the clinician in the current report 26, and adopting one or more finding codes 48 by the clinician from the suggestion pane 28 into the reporting pane 26. Optionally, in some embodiments, the one or more processors may generate and display new suggested finding codes 48 after updating the display 12/62 in the fifth step S110, and the preceding steps are repeated.

The invention has been described with reference to the preferred embodiments. Obviously, modifications and alterations will occur to others upon reading and understanding the proceeding detailed description. It is intended that the invention be construed as including all such modifications and alterations insofar as they come within the scope of the appended claims or the equivalents thereof.

1. A guided structured reporting apparatus for guiding a clinician to address unaddressed aspects of the report, the apparatus comprising:
a workstation including a display device configured to display physiological information, and an input configured to receive one or more inputs from a clinician;
one or more computer processors connected to the workstation, the one or more computer processors being configured to:
receive physiological information;
generate a display preliminary report on the display device;
receive and display finding codes from the input selected by the clinician for inclusion in the preliminary report;
generate and display suggested finding codes for potential inclusion in the preliminary report; and
update the displayed finding codes on the display device based on at least one of the clinician selected finding codes and physiological information.

2. The apparatus according to claim 1, further including at least one memory configured to store finding codes.

3. The apparatus according to claim 1, wherein the display includes:
a structured reporting panel; and
a finding code suggestion panel.

4. The apparatus according to claim 3, wherein the finding code suggestion panel includes one of a persistent finding code suggestion panel and a transient finding code suggestion panel.

5. The apparatus according to claim 3, wherein the finding code suggestion panel includes:
one or more suggested finding codes; and
one or more adoption buttons corresponding to the one or more suggested finding codes by which the clinician selects one or more of the suggested finding codes.

6. The apparatus according to claim 1, wherein the one or more suggested finding codes include a code component and a textual component.

7. The apparatus according to claim 1, wherein the one or more suggested finding codes include at least on of a binary statement finding code, a multiple-choice statement finding code, and a measurement statement finding code.

8. The apparatus according to claim 3, wherein the structured reporting panel comprises:
one or more anatomical submenus;
one or more aspect submenus;
one or more drop-down menus;
one or more suggested finding code generation buttons; and
one or more adopted finding codes.

9. The apparatus according to claim 1, wherein the one or more processors are configured to receive physiological information includes at least one of patient physiological imaging information, patient records, patient finding codes, patient structured reports, and stored finding code patterns.

10. The apparatus according to claim 1, wherein the one or more processors are configured to generate the one or more suggested finding codes using the received physiological information by at least one of a rule-driven implementation and a data-driven implementation.

11. The apparatus according to claim 2, wherein the processor is configured to generate the suggested finding codes based on an anatomical region addressed by the preliminary report and finding codes already selected for inclusion in the preliminary report.

12. The apparatus according to claim 3, wherein the display further comprises at least one of an imaging panel and a narrative report.

13. The apparatus according to claim 11, wherein the preliminary report a structured report including one or more anatomical summaries;
wherein the anatomical summaries include the textual components of the selected finding codes from a structured reporting panel.

14. A method for guiding a clinician to address unaddressed aspects of a patient report, the method comprising:
receiving physiological information at a workstation;
generating a display of a preliminary report on a display device using one or more processors;
generating and displaying one or more suggested finding codes for the clinician to select among using one or more processors;
receiving a clinician's selection of one or more of the suggested finding codes from a user input and incorporating the selected finding code in the preliminary report; and
updating the displayed finding codes based on the one or more of the clinician selected finding codes and physiological information.

15. The method according to claim 13, wherein generating the suggested finding codes includes generating a code component and a textual component.

16. (canceled)

17. The method according to claim 13, wherein the one or more suggested finding codes are generated by at least one of a rule-driven implementation and a data-driven implementation.

18. (canceled)

19. (canceled)

20. (canceled)

21. (canceled)
